(19) United States
(12) Patent Application Publication
(10) Pub. No.: US 2023/0112162 A1
(43) Pub. Date: Apr. 13, 2023

(54) COGNITIVE SCREENING METHODS

(71) Applicant: BIOGEN MA INC., Cambridge, MA (US)

(72) Inventors: Mark MOSS, Whitingham, VT (US); Victoria IRZHEVSKY, Raleigh, NC (US); Jason OSIK, Wakefield, MA (US); Joel SCHWARTZ, No. Andover, MA (US); Monroe BUTLER, Cambridge, MA (US)

(73) Assignee: BIOGEN MA INC., Cambridge, MA (US)

(21) Appl. No.: 17/904,802

(22) PCT Filed: Feb. 23, 2021
(86) PCT No.: PCT/US2021/019178

§ 371 Date: Aug. 23, 2022

Related U.S. Application Data
(60) Provisional application No. 62/980,816, filed on Feb. 24, 2020.

(51) Int. Cl. G16H 50/30; G16H 50/20

(52) U.S. Cl. CPC G16H 50/30; G16H 50/20; G16H 10/20

(57) ABSTRACT

A medical process comprising administering a digital symbol substitution test (DSST) to a patient, administering a trails marking test (TMT) to the patient after the administration of the DSST, and administering a recall test, wherein the recall test includes requesting the patient to recall digits and/or symbols from the DSST.

### CROSS-REFERENCE TO RELATED APPLICATIONS

This application claims benefit of priority from U.S. Provisional Application No. 62/980,816, filed on Feb. 24, 2020, which is incorporated herein by reference in its entirety.

### TECHNICAL FIELD

This disclosure relates generally to systems and methods of cognitively assessing patients. More particularly, at least some embodiments of the disclosure relate to methods of administering a multi-part test to identify cognitive impairment of patient.

### BACKGROUND

Cognitive assessment tools designed to identify mild cognitive impairment (“MCI”) in aged subjects are known. Examples include the Mini Mental Status Exam (“MMSE”), the Measurement of Everyday Cognition—12 item (“E-Cog-12”), and the Clinical Dementia Rating Scale (“CDR”). See, e.g. Wechsler D. Wechsler Adult Intelligence Scale Manual. New York, N.Y.: Psychological Corporation; 1955, herein incorporated by reference, and Wechsler D. Wechsler Adult Intelligence Scale—Third Edition. San Antonio: The Psychological Corporation; 1997, herein incorporated by reference. However, cognitive assessment tools specifically designed to differentiate MCI due to Alzheimer's disease (AD) from MCI caused by other non-AD conditions are limited and time consuming. Methods for screening MCI subjects for clinical trials with AD biomarkers, for example positron emission tomography (“PET”) or cerebrospinal fluid (“CSF”), are costly and present increased risks to the safety of the patient. There is an industry need for a brief and more sensitive cognitive screening tool that may be used to identify indicators of MCI due to AD, e.g., β-Amyloid burden.

### SUMMARY OF THE DISCLOSURE

According to an example, a medical process may comprise administering a digital symbol substitution test (DSST) to a patient, administering a trails marking test (TMT) to the patient after the administration of the DSST, and administering a recall test, wherein the recall test includes requesting the patient to recall digits and/or symbols from the DSST.

In another example, the medical process may be completed within approximately 10 minutes. Administering the DSST, the TMT, and/or the recall test may be performed digitally. Administering the DSST may last approximately two minutes. Administering the recall test may be within six minutes of completion of administering the DSST.

According to another example, the medical process may further comprise assessing the patient for one or both of mild cognitive impairment and a presence of β-amyloid burden, based on results of administering the DSST, the TMT, and the recall test. The assessing step may account for no other cognitive test administered to the patient. The medical process may further comprise determining one or more demographic characteristics of the patient. The demographic characteristics may include age, education, gender, and genetic disposition, wherein the genetic disposition includes the presence of a ApoE genotype. The assessing step may account for one or more of the demographic characteristics. The assessing step may be based on only the results of administering the DSST, the TMT, and the recall test. Administering the TMT may include administering a first portion and a second portion, and administering the recall test may be immediately after completion of administering the TMT. Administering the recall test may further include requesting the patient to recall as many digits or symbols as possible from the DSST.

In another example, no cognitive test may be administered to the patient between administering the DSST and administering the TMT, and between administering the TMT and administering the recall test. Administering the TMT may include administering a first portion and a second portion.

It may be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate exemplary embodiments of the present disclosure and together with the description, serve to explain the principles of the disclosure.

FIG. 1A is a chart illustrating a method according to this disclosure.

FIGS. 1B and 1C are exemplary cognitive impairment tests.

FIG. 2A is a chart illustrating results of an exemplary method according to this disclosure.

FIG. 2B is a chart illustrating results of a method known in the art.

FIGS. 2C-2F are charts illustrating results of an exemplary method according to this disclosure.

FIG. 3 is a chart illustrating results of another exemplary method.

FIGS. 4A-4B are charts comparing an exemplary method according to this disclosure to methods known in the art.

### DETAILED DESCRIPTION

This disclosure is drawn to systems and methods for cognitively assessing patients for MCI and whether said MCI is attributable to AD, among other aspects. Reference will now be made in detail to aspects of the disclosure, examples of which are shown in the accompanying figures and further discussed below. Wherever possible, the same or similar reference numbers will be used through the drawings to refer to the same or like parts.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, the term “exemplary” as used herein is used in the sense of “example,” rather than “ideal.”

Embodiments of the disclosure may solve one or more of the limitations in the art. The scope of the disclosure, however, is defined by the attached claims and not the ability to solve a specific problem.

Disclosed herein are methods for implementing a cognitive screen to probe neuropsychological function in circuits vulnerable to early AD neurodegeneration, including measures of episodic memory, incidental learning, fluency, processing speed and executive function. The disclosed cognitive screen method, which may be administered within a certain duration of time, e.g., 10 minutes, predicts β-amyloid PET status in patients with MCI with greater sensitivity and specificity than standard cognitive and functional endpoints, such as MMSE, E-Cog-12, and CDR. The screen also exhibits either superior accuracy or non-inferiority with shorter evaluation time in some cases at predicting β-amyloid compared to standard measures irrespective of ApoE genotype. The disclosed targeted screening approach alleviates the time burden of neuropsychological testing, demonstrates ease of use, and could be adapted for patient screening in naturalistic settings. Cognitive data were assessed from the screening phase of a clinical study designed to identify amyloid-related imaging abnormalities (ARIA) associated with treatment of Aducanumab in patients with MCI and early dementia.

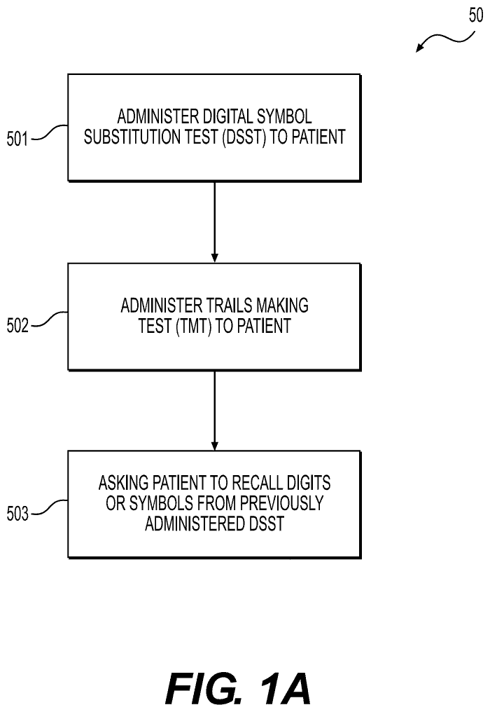

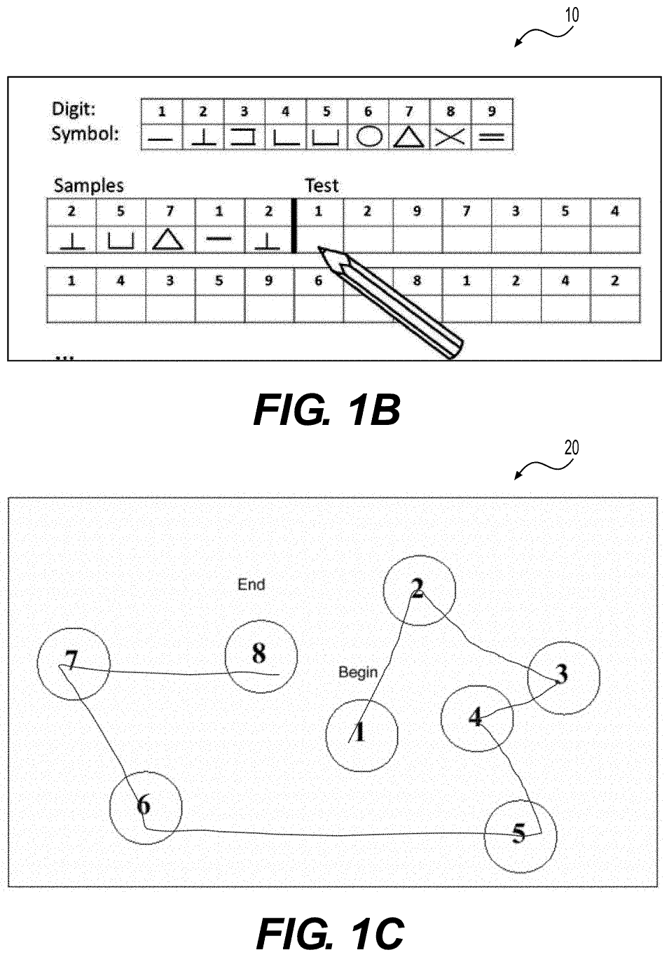

Embodiments of this disclosure relate to a screening tool for identifying mild cognitive impairments. Embodiments of this disclosure relate to systems and methods for identifying the presence of β-amyloid burden in MCI and early dementia. Referring to FIGS. 1A-1C, an exemplary cognitive screening method 50 is further discussed. As can be seen, method 50 includes the following steps:
Step 501 includes administering the Digit Symbol Substitution Test (DSST) to a patient (an example of DSST being shown in FIG. 1B), where the patient writes (by hand on paper or digitally via a tablet or other computer) as many symbols, corresponding to digits, as possible within a relatively short time frame, in some examples, approximately two minutes.
Step 502 includes administering the Trails Making Test (TMT) Part A & B to the patient (an example of TMT being shown in FIG. 1C), wherein the test may require giving a patient a piece of paper (or a tablet or other computer) with a plurality of randomly placed numbered circles as Part A (in some examples, eight circles are used), and where the patient then draws a line between the circles in sequential order from 1 until the last circle. In examples, eight circles are used, though more or less numbers of circles (and different shapes) may be used.
Step 503 includes, upon completion of the Trails Making Test, asking the patient to recall as many digits or symbols as possible from said DSST. Preferably, asking the patient to recall as many digits/symbols as possible within approximately six minutes of completion of the DSST. In an example, the test administrator may ask the patient orally to provide the digit (number) that corresponds to a stated or shown symbol. In another example, the test administrator may ask the patient orally to provide the symbol that corresponds to a stated or shown digit. The time period between the DSST and the this recall portion of the test may be other than six minutes, for example, the time period may be two, three, four, five, seven, eight, nine, or more minutes. It is noted that any suitable filler task may be incorporated to ensure that the recall task administered in step 503 is sufficiently distant in time from DSST of step 501. For example, a filler task such as a same-different picture comparison test may be administered to fill in any time difference between completion of TMT of step 502 and initiation of the DSST recall test. Any such filler task, however, is not used in assessing a state of the patient.

The entirety of screening method 50 may be completed by the patient within a relatively short amount of time, for example, within about 10 minutes, but not limited thereto.

Both DSST and TMT, examples of which are shown in FIGS. 1B and 1C, are known cognitive impairment tests. Supra Wechsler, Wechsler Adult Intelligence Scale Manual. They may be hand drawn tests—pen to paper, or can be done digitally on a tablet, for example. The Digit Symbol Substitution Test (DSST) is a known test used in cognitive screening. See Moss M B et al. (2007). “Neuropsychological measures in normal individuals that predict subsequent cognitive decline”. Archives Neurology. 64: 862-871, incorporated herein by reference. DSST tests the cognitive domains of psychomotor speed and memory. The Trails Making Test (TMT) Part A&B is also a known cognitive screening test. See Tombaugh T N (2004). “Trail Making test A and B: Normative Data Stratified by Age and Education”. Archives of Clinical Neuropsychology. 19 (2): 203-214, incorporated herein by reference. TMT tests the cognitive domains of executive function (set-shifting) and psychomotor speed. However, both DSST and TMT are typically included in a cognitive test battery, including tens or hundreds of additional assessment tests, all of which may be administered to a patient for assessment purposes. Moreover, in said test battery, DSST and TMT may not be administered in the order discussed above, and, in some instances, may be separated by a number of tests in between. The disclosed method, e.g., method 50, requires only the administration of DSST, then TMT subsequently thereafter, and then involves asking patients, after they complete the Trails Making Test Part A&B, to recall digits/symbols from the DSST.

Based on the results of test method 50, a prediction regarding the β-amyloid PET status of the patient is made. It has been discovered that the combination of only these two tests (DSST and TMT), plus the recall of the digits/symbols separated from the DSST by a particular time period, is short and accurate/sensitive to finding mild cognitive impairment and the presence of this protein that is symptomatic of Alzheimer's. Thus, the disclosed method is more streamlined relative to typical cognitive test batteries, and may be as effective or more effective than said test batteries, as further discussed below.

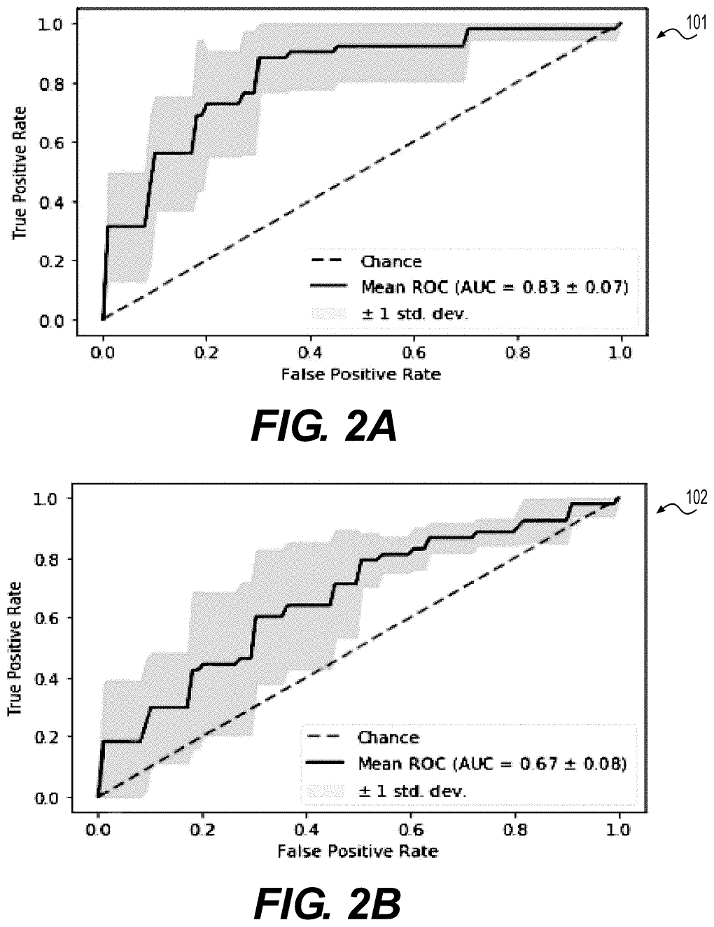

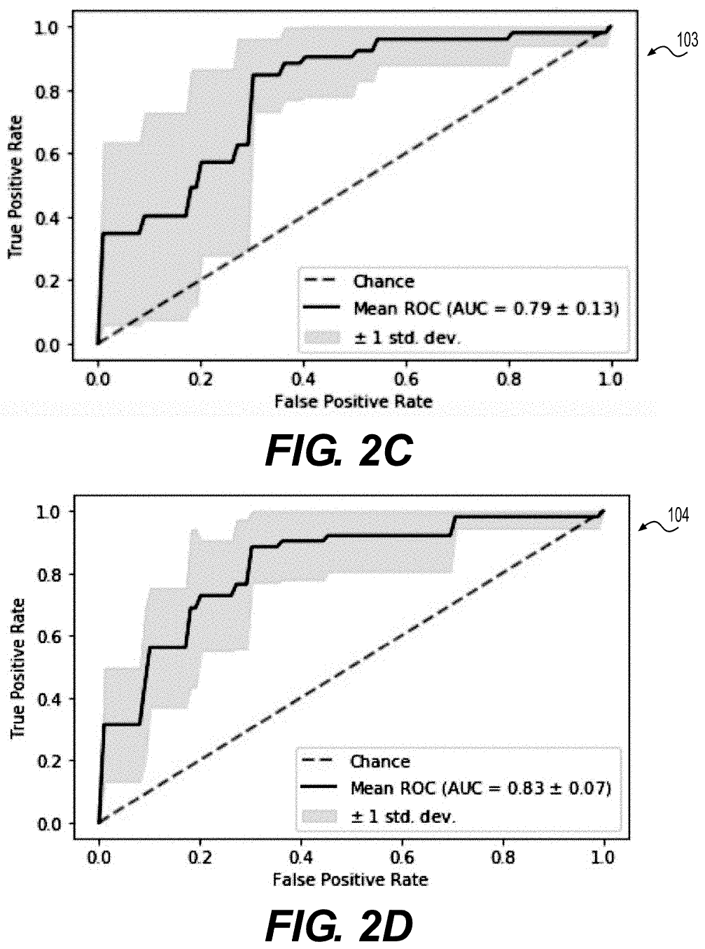

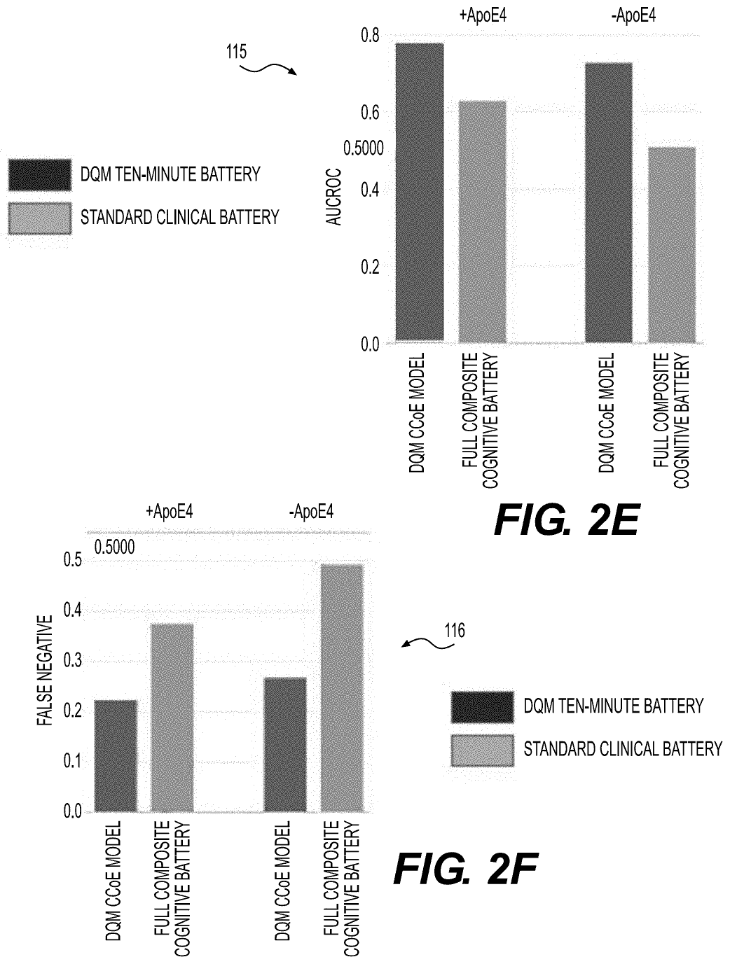

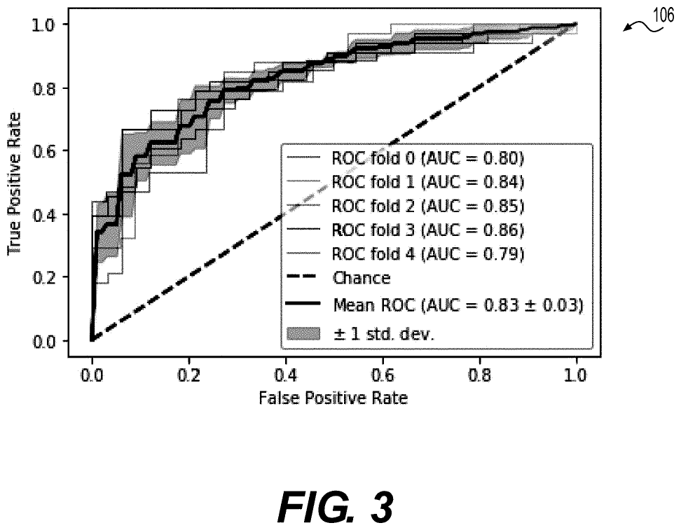

As shown in FIGS. 2A-3, the disclosed method has greater sensitivity and specificity (at standard 0.5 threshold) than standard cognitive tests such as the MMSE, E-Cog-12, and CDR. The test is more accurate than standard screens at predicting β-Amyloid, based on the plots discussed below, using the area under the receiver operating characteristic (ROC) curve (AUC of ROC) method for accuracy determination. The disclosed approach also alleviates the burden of neuropsychological testing, is easy to use, and may be adapted for patient screening in naturalistic settings.

### Presence of β-Amyloid Burden

To obtain the data presented in FIGS. 2A-2F, a study (the EVOLVE study) was conducted administering method 50 of FIG. 1A. “A Ten-Minute Cognitive Screening Tool that Identifies the Presence of β-Amyloid Burden in MCI and Early Dementia: Data from the EVOLVE Safety Trial,” Moss et al. Science Day 2020, February 24, 2020, incorporated herein by reference (and attached as Appendix 1 in U.S. Provisional Application No. 62/980,816, filed on Feb. 24, 2020, which is incorporated herein by reference in its entirety). The study compares AUC-ROC performance of an exemplary assessment, e.g., method 50, vs. traditional cognitive and functional measures to predict β-Amyloid PET status in a representative population of aged patients with MCI of unclear etiology.

Tested patients included 78 subjects (35 females, 43 males) with MCI with unclear etiology, in which the mean age was 70.5 years, and SD was 7.9. The mean education was 15.7 years, and SD was 3.3. Regarding genetic disposition, e.g., ApoE genotype, there were 40 ApoE₄ carriers, and 38 non-ApoE₄ carriers. 53 patients were positive and 25 patients were negative from amyloid PET.

The standard functional and cognitive tests included those typically administered—Mini Mental Status Exam (MMSE), Measurement of Everyday Cognition—12 item (E-Cog-12), and the Clinical Dementia Rating Scale (CDR). An exemplary cognitive screening, e.g., method 50, was also administered within a ten minute period. After administration of both the standard tests and the ten minute screening, results were recorded and plotted, as shown in FIGS. 2A-2F. To produce said plotting, a regularized logistic regression model was used to evaluate the ten-minute screening against a composite of traditional endpoints, based on the AUC of ROC. The value of the AUC of ROC indicates the diagnostic ability of the screening; an AUC of 1 indicates all true positive results are captured by the screening.

FIG. 2A illustrates plot 101, the mean AUC of ROC from 100 iterations of a stratified 5-fold cross-validation (CV) with data imputed from the scores of the ten-minute screening, while also taking into account ApoE (genetic disposition). As shown, plot 101 demonstrates an AUC of 0.83 +/−0.07. FIG. 2B illustrates a plot 102 illustrating the AUC of ROC of the standard screening including MMSE, E-Cog-12, and CDR, while also taking into account ApoE. Plot 102 demonstrates an AUC of 0.67 +/−0.08. Thus, the ten-minute screening demonstrated significant improvement in accurate screening/diagnostic ability over the standard screening that was administered.

Additional plot 103 of FIG. 2C and plot 104 of FIG. 2D illustrating the AUC of ROC of the ten-minute screening, e.g., method 50, were recorded, while taking into consideration other parameters, e.g., age, ApoE. Plot 103 takes into account age, but not genetic disposition. As shown, plot 103 demonstrates an AUC of 0.79 +/−0.13. Plot 104 takes into account age and genetic disposition. As shown, plot 104 demonstrates an AUC of 0.83 +/−0.07. Thus, the ten-minute screening, while taking into account other parameters, demonstrates accurate screening/diagnostic ability, as indicated by an AUC of at least 0.79. In addition, plot 103 indicates the accuracy of test 50 without considering genetic disposition. This shows the value of test 50 in settings where genetic disposition is unknown or difficult to obtain.

Referring to FIGS. 2E and 2F, a chart 115 provides a comparison between the resulting AUC of ROC from the ten-minute screening vs. the AUC of ROC from administering a standard full composite cognitive battery in the EVOLVE study, which included CDR-SB, MMSE, and ECog. As shown in chart 115, the ten-minute screening demonstrated superior screening ability (measured via AUC-ROC) whether or not genetic disposition, ApoE, was taken into consideration. A chart 116 (FIG. 2F) provides a comparison between the resulting false negatives from the ten-minute screening vs. the false negatives resulting from the standard full composite cognitive battery. Again, as shown in chart 116, the ten-minute screening demonstrated less likelihood of diagnosing a false negative whether or not genetic disposition, ApoE, was taken into consideration.

Furthermore, it is noted that the performance of method 50 generalizes to other larger datasets/studies in the field, e.g., The Swedish Biofinder Study. The Swedish Biofinder Study administered to patients (a total of about 100 MCI participants) a cognitive assessment test, significantly more extensive relative to method 50. For example, the Study included a plurality of different cognitive tests, such as MMSE, 10-word list delayed recall (ADAS-cog), A Quick Test of cognitive speed (AQT), Verbal fluency, Clock Drawing Test (CDT), cube-copying test, the Stroop Test, and months backwards, each test being selected to measure different cognitive functions. “Clinical Evaluation of Participating Individuals Included in the Biofinder Cohorts,” The Swedish Biofinder Study (available online at https://biofinder.se/data-biomarkers/clinical-evaluation/), incorporated by reference herein. Moreover, the Swedish Biofinder Study also takes into account various factors, including demographics, e.g., age, education, gender, cognitive data, biomarkers including amyloid PET, MRI, CSF, and plasma, and genetic disposition including ApoE and PRS, among many other factors and tests (including those listed above). Said study included, amongst its extensive battery of tests, DSST (120 seconds), TMT Parts A (150 seconds) and B (300 seconds), and a comparable learning/memory test to DSST paired recall, e.g., ADAS-Cog Recall (60 seconds). The Swedish Biofinder Study (available online at https://biofinder.se/), incorporated by reference herein.

To illustrate the generalization of method 50 to said Biofinder dataset, results of DSST, TMT, and the comparable recall test in the Biofinder Study were extracted from the data resulting from the battery of tests. Said extracted results were used to determine the sensitivity/specificity of DSST, TMT, and the comparable recall in predicting the presence of β-amyloid burden, as could otherwise be observed via PET. This determination is illustrated in plot 106 of FIG. 3. Plot 106 of FIG. 3 illustrates the mean AUC of ROC from 100 iterations of a stratified 5-fold cross-validation (CV) with imputed data from a select portion of the Biofinder Study discussed above. As shown, plot 106 demonstrates an AUC of 0.83 +/−0.03, similar to the AUC ROC measured of the ten-minute screening in the EVOLVE study, as shown in plot 101 of FIG. 2A. Thus, such data further affirms the effectiveness of method 50 in larger datasets and settings, and the improvement method 50 provides in predicting β-amyloid burden in MCI patients.

### MCI Screening

In addition to predicting β-amyloid burden of MCI patients accurately, data from various studies, e.g., Alzheimer's Disease Neuroimaging Initiative (ADNI) and The Swedish Biofinder Study, demonstrate the ability of method 50 to screen individuals for MCI effectively.

Both the ADNI study and Biofinder Study administered cognitive assessment tests to MCI individuals and non-MCI individuals. The ADNI study administered to 582 participants (about 374 participants having MCI) a battery of cognitive tests, significantly more extensive relative to method 50. The battery of cognitive tests in the ADNI study included ADAS-COG13, American National Adult Reading Test, category fluency tests, clock drawing test, Cogstate Brief Battery, Logical Memory, MMSE, MOCA, Multi-Lingual Naming Test, RAVLT, and other additional tests. Amongst the plurality of tests in the battery, the ADNI study also administered DSST, TMT Parts A and B, and a comparable learning/memory test to DSST paired recall, e.g., RAVLT. “Alzheimer's Disease Neuroimaging Initiative 3 (ADNI3) Protocol,” ADNI Protocol v.1.0, May 24, 2016, incorporated by reference herein. The ADNI study took into consideration other factors including demographics including age, gender, education, biomarkers including amyloid PET, and genetic disposition, e.g., ApoE genotype. The Biofinder Study administered to a total of 428 participants (about 100 participants having MCI). As discussed above, the Biofinder Study also included, amongst its extensive battery of tests, DSST, TMT Parts A and B, and a comparable learning/memory test to DSST paired recall, e.g., ADAS-Cog Recall.

To illustrate the MCI screening ability of method 50, results of DSST, TMT, and a comparable recall test in the ADNI study and the Biofinder Study were extracted from the larger datasets from those studies. Said extracted results were used to compare method 50, e.g., DSST, TMT, and memory recall, to MMSE in screening for MCI, along various metrics.

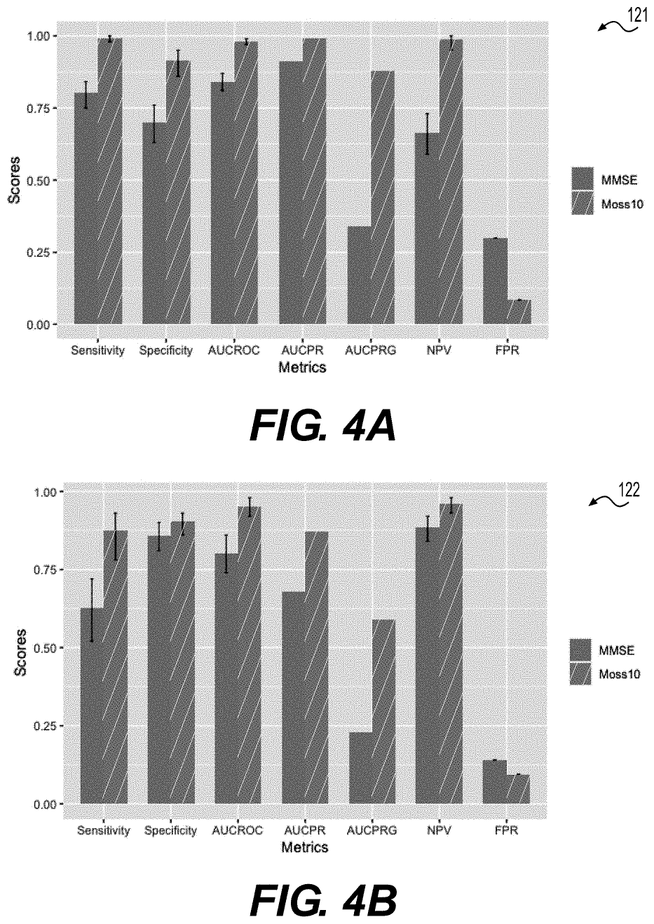

Referring to FIG. 4A, a chart 121 illustrates a comparison between method 50 (referred to as Moss 10) and MMSE as a MCI screen, from the ADNI dataset. MCI screening was conducted with age-matched controls, and without consideration of ApoE presence. As shown, method 50 outperforms MMSE in screening for MCI across various metrics, including sensitivity, specificity, AUC ROC, AUC Precision Recall (PR), AUC Precision Recall Gain (PRG), negative predictive value (NPV), and False Positive Rate (FPR), according to the ADNI dataset. The error bars shown in chart 121 are of 95% confidence intervals.

Referring to FIG. 4B, a chart 122 similarly illustrates a comparison between method 50 (referred to as Moss 10) and MMSE as a MCI screen, from the Biofinder dataset. Again, MCI screening was conducted with age-matched controls, and without consideration of ApoE presence. As shown, method 50 outperforms MMSE in screening for MCI across various metrics, including sensitivity, specificity, AUC ROC, AUC Precision Recall (PR), AUC Precision Recall Gain (PRG), NPV, and False Positive Rate (FPR), according to the Biofinder Study dataset as well. The error bars shown in chart 122 are of 95% confidence intervals.

In view of the above, larger datasets affirm that method 50 is able to provide an improved MCI screening relative to typical cognitive assessment tests, e.g., MMSE, based on a plurality of metrics.

### Computer Implementation

A computer may be configured to execute techniques described herein, according to exemplary embodiments of the present disclosure, e.g., method 50. Specifically, the computer (or “platform” as it may not be a single physical computer infrastructure) may include a data communication interface for packet data communication. The platform may also include a central processing unit (“CPU”), in the form of one or more processors, for executing program instructions. The platform may include an internal communication bus, and the platform may also include a program storage and/or a data storage for various data files to be processed and/or communicated by the platform such as ROM and RAM, although the system may receive programming and data via network communications. The system also may include input and output ports to connect with input and output devices such as keyboards, mice, touchscreens, monitors, displays, sensors, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a general or special purpose computer and/or data processor that is specifically programmed, configured, and/or constructed to perform one or more computer-executable instructions for implementing the disclosed methods. While aspects of the present disclosure, such as certain functions, may be described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

It will be apparent to those skilled in the art that various modifications and variations can be made to the disclosed systems and methods without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A medical process, comprising:
administering a digital symbol substitution test (DSST) to a patient;
administering a trails marking test (TMT) to the patient after the administration of the DSST; and
administering a recall test, wherein the recall test includes requesting the patient to recall digits and/or symbols from the DSST.

2. The medical process of claim 1, wherein the medical process is completed within approximately 10 minutes.

3. The medical process of claim 1, wherein administering the DSST, the TMT, and/or the recall test is performed digitally.

4. The medical process of claim 1, wherein administering the DSST lasts approximately two minutes.

5. The medical process of claim 1, wherein administering the recall test is within six minutes of completion of administering the DSST.

6. The medical process of claim 1, further comprising assessing the patient for one or both of mild cognitive impairment and a presence of β-amyloid burden, based on results of administering the DSST, the TMT, and the recall test.

7. The medical process of claim 6, wherein the assessing step accounts for no other cognitive test administered to the patient.

8. The medical process of claim 6, further comprising determining one or more demographic characteristics of the patient.

9. The medical process of claim 8, wherein the demographic characteristics include age, education, gender, and genetic disposition, wherein the genetic disposition includes the presence of a ApoE genotype.

10. The medical process of claim 9, wherein the assessing step accounts for one or more of the demographic characteristics.

11. The medical process of claim 6, wherein the assessing step is based on only the results of administering the DSST, the TMT, and the recall test.

12. The medical process of claim 1, wherein administering the TMT includes administering a first portion and a second portion, and administering the recall test is immediately after completion of administering the TMT.

13. The medical process of claim 1 wherein administering the recall test further includes requesting the patient to recall as many digits or symbols as possible from the DSST.

14. The medical process of claim 1, where no cognitive test is administered to the patient between administering the DSST and administering the TMT, and between administering the TMT and administering the recall test.

15. The medical process of claim 1, wherein administering the TMT includes administering a first portion and a second portion.
